(19) United States
(12) Patent Application Publication
(10) Pub. No.: US 2020/0367765 A1
(43) Pub. Date: Nov. 26, 2020

(54) METHOD AND SYSTEM FOR MEASURING BLOOD FLOW

(71) Applicant: Siemens Healthcare GmbH, Erlangen (DE)

(72) Inventors: Mario Bacher, Erlangen (DE); Peter Speier, Erlangen (DE)

(21) Appl. No.: 16/881,243

(22) Filed: May 22, 2020

(30) Foreign Application Priority Data
May 24, 2019 (EP) 19176395.2

(51) Int. Cl. A61B 5/0265; A61B 5/00; G01R 33/563

(52) U.S. Cl. CPC A61B 5/0265; A61B 5/7228; A61B 2562/0223; G01R 33/563; A61B 5/0033

(57) ABSTRACT

A method for measuring blood flow is provided. A modulated magnetic field is generated by a generator coil. Response signals of the modulated magnetic field are record from an object with an RF reception system. The response signals are measured by at least two RF antennae of the RF reception system. A flow component of the recorded response signals is separated from signal components resulting from movements of the object.

### CROSS REFERENCE TO RELATED APPLICATIONS

This patent application claims the benefit of EP 19176395.2 filed May 24, 2019, which is hereby incorporated in its entirety

### FIELD

Embodiments provide a method and a system for measuring blood flow for a magnetic resonance imaging system (“MRI system”).

### BACKGROUND

MRI systems are used in medical examinations for recording (imaging) data of an examination object by exciting its nuclear spins that are aligned within a strong basic magnetic field. The precession or relaxation of the spins from the excited state into a state with less energy generates, as a response, a magnetic alternating field (radio frequency signal, “RF signal”) that is received via RF antennae. The RF frequency strongly depends on the basic magnetic field.

Depending on the pulse sequence used for recording, the measurement of the MRI system requires a number of milliseconds up to a number of seconds. While a longer recording time usually results in minimal noise artifacts, the influence of motion artifacts increases with the duration of the measurement.

Although most patients try to remain still, in order to avoid motion artifacts, there are unavoidable movements of the patient that cannot be stopped, such as e.g. breathing or heartbeat.

Conventionally, the movements of a patient are measured by using mechanical sensors or electrodes that measure the excitation potential of the muscles.

German patent application 10 2015 203 385 A1 describes a basic method of capturing the movements using a high-frequency signal. The signal is permanently captured in a patient recording of a magnetic resonance tomography system, and signal changes due to movements (e.g., due to changing interferences or damping) are evaluated. A movement of the patient caused by breathing or heartbeat may then be identified from certain patterns of the signal.

German patent application 10 2015 224 158 A1 discloses a special transmitter for pilot tone navigation in a magnetic resonance tomography system. The pilot tone navigator includes a power supply and an antenna. The transmitter is configured to emit a pilot tone signal in the form of an electromagnetic alternating field by way of the antenna. The transmitter includes a decoupling element that decouples the transmitter output of signals that the antenna in a magnetic resonance tomography system receives by excitation pulses of the magnetic resonance tomography system.

German patent application 10 2015 224 158 A1 further discloses a method of identifying a movement of a patient using a magnetic resonance tomography system with such transmitter that is arranged in close proximity to the heart or the lungs of the patient (e.g., on a body surface of the patient at a minimal distance from the organs). Then, the transmitter transmits the pilot tone signal and the magnetic resonance tomography system receives the pilot tone signal with one or a number of antennae and receivers that are typically provided to receive a magnetic resonance signal.

Although it is possible to measure the motion of the heart or of the lungs, no non-invasive sensor currently exists that is capable of continuously detecting flow within a magnetic resonance (“MR”) scanner environment. This is very disadvantageous, since the accurate detection of blood flow is an important factor in non-contrast MR angiography (NC-MRA) like NATIVE or QISS of extremities where the correct trigger time, i.e. the arrival of the pulse wave in the examination volume, is delayed with respect to the ECG R-peak by an unknown time.

The unknown time delay between contraction of the heart and arrival of the pulse wave in the imaging volume is up to now either estimated based on length measurements or trial scans, or free-running sequences are used to measure continuously.

Currently existing magnetic flowmeters for blood flow use electrodes attached to the skin to measure induced potentials due to motion.

### BRIEF SUMMARY AND DESCRIPTION

The scope of the present disclosure is defined solely by the appended claims and is not affected to any degree by the statements within this summary. The present embodiments may obviate one or more of the drawbacks or limitations in the related art.

Embodiments provide systems, devices, and methods for facilitating an improvement for measurements of a blood flow of a patient.

Embodiments provide a method for measuring blood flow. The blood flow may be the blood flow in an object of a patient, where the object is a blood vessel, but may also be regarded as an organ including blood vessels or another cavity where blood may flow (also a wound). The patient may be positioned in a magnetic resonance imaging system.

The method for measuring blood flow includes the following steps:
Generating a (low-power) modulated magnetic field by a generator coil. The field may include at least one frequency that may be measured by an RF reception system. A simple modulated magnetic field may include the shape of a sine wave. However, it is also possible to include a more complex modulation including a multiplicity of sine waves, e.g. a rectangular modulation. Since it is advantageous in magnetic resonance imaging to use the RF reception system of a magnetic resonance scanner, the frequency (or at least one dominant frequency) may accord to the receiving bandwidth of the RF reception system but deviate from the Larmor frequency recorded during regular data acquisition for imaging. Best results may be achieved when the modulated magnetic field is sent directly to the object positioned for examination.
Recording response signals of the modulated magnetic field from the object with the RF reception system. The response signals are measured by at least two RF antennae of the RF reception system. The RF antennae may be coils, for example coils used for imaging in a magnetic resonance scanner, however, the RF antennae may also have another shape (e.g. of a linear dipole).
Separating a flow component of the recorded response signals from signal components resulting from movements of the object. Such movements may include besides muscular movements of the extremities, cardiac and respiratory motion or bowel movements. A movement-dependent change in the received signal may be identified and then separated by using signal analysis. For example, a flow movement may be detected by identifying a variation in the amplitude of the modulated magnetic field (“pilot tone signal”), caused by changes in the load of the RF antennae or the change in superimpositions or damping due to the blood flow in a blood vessel. The flow movement may be differentiated from other disturbing influences by the specific frequency and signal profile that is different from the frequency or signal profile of e.g. a respiratory movement or a heartbeat.

Embodiments provide a system for measuring blood flow that includes the following components:
A first data-interface configured for sending a signal to a generator coil. The signal is configured to generate a (low power) modulated magnetic field in the generator coil. The signal may be generated by a signal generator that may also be part of the system. However, the signal generator may also be part of an (external) generator coil, wherein the signal sent through the data interface may be a simple “on” signal to trigger the signal generator.
A second data-interface configured for recording response signals of the modulated magnetic field from an object measured with an RF reception system. At least the response signals are measured by two RF antennae of the RF reception system. The RF reception system may be part of the system and may include data acquisition units that are configured to modify the signals of the RF antennae, e.g. amplify and/or shape it, such that the system may use the signals for further processing. For example, the RF reception system may provide digital data of the recorded signals. It may be noted that the first and second data interface may be one single unit configured for bi-directional communication.
A separation unit configured to separate a flow component of the recorded response signals from the signal components resulting from movements of the object.

The whole setup with the system including the generator coil RF reception system may be designated as “Pilot Tone navigator system” since it uses a pilot “tone” (the modulated magnetic field) for measurements. Changes in the electromagnetic environment within the examination volume, e.g. by blood flow, respiratory motion, cardiac motion or further muscular or bowel movements, alter the generated modulated magnetic field. When the generative field is set to a frequency just outside the imaging bandwidth around the Larmor frequency, the modulated magnetic field may easily be detected by the RF antennae.

The generator coil may include a transmitter for pilot tone navigation in a magnetic resonance tomography system. The generator coil generates a modulated magnetic field with a first frequency band. An excitation signal is generated by an RF transmission antenna system of a magnetic resonance imaging system to generate a magnetic resonance signal with a second frequency band from the object. The second frequency band is at least essentially outside the first frequency band. The at least two RF antennae of the RF reception system are configured to receive a frequency band that includes the first frequency band and the second frequency band.

Since blood is a (para)magnetic fluid, the modulated magnetic field induces eddy currents in the flowing blood, that in turn generates a magnetic field opposing the inducing field. Thus, when the blood moves due to the heart's pumping activity, an additional current is induced due to the motion that changes the local magnetic environment and the response would differ from the original “tone”, i.e. the original modulation of the initial magnetic field.

Since the response is measured using a (e.g. relatively vast) number of RF antennae (e.g. receiver coils of an MR scanner), the flow component may be separated from the components induced from cardiac and respiratory motion or bowel/muscular movements using appropriate blind and/or semi-blind source separation methods such as e.g. Independent Component Analysis (ICA) or training with ground truth data.

Embodiments provide a control device for controlling a magnetic resonance imaging system that includes a system and/or is configured to perform a method for measuring blood flow. The control device may include additional units or devices for controlling components of a magnetic resonance imaging system, e.g. a sequence control unit for measurement sequence control, a memory, an RF transmission device that generates, amplifies, and transmits RF pulses, a gradient system interface, an RF reception device to acquire magnetic resonance signals and/or a reconstruction unit to reconstruct magnetic resonance image data.

Embodiments provide a magnetic resonance imaging system including a control device or that is at least configured to perform a method for measuring blood flow.

Some units or modules of the system or the control device mentioned above may be completely or partially realized as software modules running on a processor of a system or a control device. A realization largely in the form of software modules may include the advantage that applications already installed on an existing system may be updated, with relatively little effort, to install and run the units of the present application. Embodiments provide a computer program product with a computer program that is directly loadable into the memory of a device of a system or a control device of a magnetic resonance imaging system, and that includes program units to perform the steps of the method for measuring blood flow when the program is executed by the control device or the system. In addition to the computer program, the computer program product may also include further parts such as documentation and/or additional components, also hardware components such as a hardware key (dongle etc.) to facilitate access to the software.

A computer readable medium such as a memory stick, a hard-disk or other transportable or permanently-installed carrier may serve to transport and/or to store the executable parts of the computer program product so that these may be read by a processor unit of a control device, an MPSU or a system. A processor unit may include one or more microprocessors or their equivalents.

In a method for measuring blood flow, the generator coil is embedded in a magnetic resonance scanner of a magnetic resonance imaging system, for example, near or in an RF reception antenna system (with one or more RF antennae) of the magnetic resonance scanner. The generator coil may be positioned adjacent the examination space or in a unit that is placed at, under or on a patient for examination, e.g. in a local coil setup, since the nearer the generator coil is positioned to the patient the greater the excitation of the desired area with the modulated magnetic field. A number of coils used for imaging in a magnetic resonance scanner may be used as generator coils.

The RF reception system used for recording the response signals of the modulated magnetic field may be the RF reception system of a magnetic resonance imaging system. The response signals of the modulated magnetic field may be recorded by RF antennae from the RF reception antenna system of the magnetic resonance scanner. The, or at least one, (dominant) frequency of the modulation of the magnetic field may lie within the normal bandwidth of the RF reception antenna system of the magnetic resonance scanner (e.g. in the order of some ten or hundred MHz). The frequency is dependent of the field strength, e.g. at a basic magnetic field of 7 T the frequency may be close to 300 MHz, at field strength below 0.5 T the frequency may be lower than 20 MHz. At a basic magnetic field of 1.5 T the frequency may be especially over 22 MHz and/or lower than 150 MHz. For example, since the Larmor frequency at a basic magnetic field of 1.5 T is about 62.5 MHz, the frequency used for modulation may be between 57 MHz and 61 MHz or 64 MHz and 68 MHz. More than three RF antennae may be used for measurement, since the more antennae are used the better the separation of the response signals may be performed.

With the Larmor frequency for a proton with the gyromagnetic moment of 267.5 MHz/T, the Larmor frequency fL at a magnetic field B may be calculated from fL=267.5/(2·π)·B MHz/T. Thus, the frequency f used for modulation at a present magnetic field B may be around (but especially not equal) fL=42.58·B MHz/T. The frequency f used for modulation at a present magnetic field B may be below 42·B MHz/T and above 43·B MHz/T, however, the frequency f used for modulation at a present magnetic field B may be above 38·B MHz/T (if lower than the Larmor frequency) or below 47·B MHz/T (if higher than the Larmor frequency).

In a method for measuring blood flow, the separation of the flow component of the recorded signals from the signal components resulting from movements of the object, e.g. cardiac and respiratory motion, is achieved by using (appropriate) blind and/or semi-blind source separation methods, for example, by using Independent Component Analysis (ICA) or by a machine learning algorithm trained with ground truth data. The methods for separation of signal contributions are known. The method for measuring blood flow may include recording a (image) navigator during a reference scan and determining a combination that provides a norm (e.g. L2) that minimizes the difference between the navigator and the recorded signal (of the response signal or of the flow component).

In a method for measuring blood flow, the object is arranged between the generator coil and the RF antennae. At least the signals from the two RF antennae of the RF reception system that are positioned closest to a line parallel to the blood flow intended to be measured is recorded. Since the course of blood vessels in a body is known and the region of interest is known, the appropriate antennae (e.g. coils of a magnetic resonance scanner) may be selected accordingly. Alternatively, it is also possible to arrange antennae appropriately.

The object may be between the generator coil and the RF antennae, since there may be also recorded a useful response signal if an antenna array is used with an integrated generator coil. The magnetic field emitted by the object may be measured all around the object. The response signals of the modulated magnetic field may be measured at an arbitrary position around the object and not only at a position opposite the generator coil.

In a method for measuring blood flow, a modulation of the modulated magnetic field includes a frequency within a frequency range near the Larmor frequency of the hydrogen atoms of the object in an applied magnetic field, for example, within a variance of less than 10 percent deviating from the Larmor frequency.

Alternatively or additionally, a modulation of the modulated magnetic field includes a frequency that is within the receiving frequency bandwidth of the RF antennae used for the measurement, for example within a frequency range of less than 10 percent deviating from the Eigenfrequency of the RF antennae.

In a method for measuring blood flow, after separating the flow component of the recorded signals from the signal components resulting from movements of the object, parameters of the flow are computed, for example a value of a flux and/or a periodicity. This may be achieved with a computing unit configured to compute parameters of the measured flow, for example a value of a flux and/or a periodicity. For example, the periodicity may be derived from the shape of the recorded signal over time, the flux may be derived from the delay of recorded signals from different RF antennae.

A system includes a generator coil configured to generate a modulated magnetic field. The generator coil may be positioned inside a magnetic resonance scanner. Additionally, the system may include a generator unit for producing the modulation signal for the modulated magnetic field.

The generator coil may be dumbbell-shaped, e.g. with two circular-shaped parts and an elongated part between the circular-shaped parts. The circular-shaped parts may be small relative to the wavelength of the modulated magnetic field. The parts may include a diameter between 5 mm and 10 mm and the elongated part a length between 10 mm and 50 mm. The maximum field strength of the modulated magnetic field lies in the region of mT and is typically below 100 mT and above 1 mT. When receiving units of an MR scanner are used, the field strength may be adjusted such that the received signal level is in the region of the signal levels received at normal MR imaging so that there is no overdrive of the electronics.

A system includes an RF reception system with at least two RF antennae configured to record response signals of the modulated magnetic field from the object. At least the response signals are measured by the RF antennae. The RF antennae may be an RF reception antenna system of a magnetic resonance imaging system.

### BRIEF DESCRIPTION OF THE FIGURES

FIG. 1 depicts a simplified MRI system with an example for a system according to an embodiment.

FIG. 2 depicts a block diagram of the process flow of a method for measuring blood flow according to an embodiment.

FIG. 3 depicts an example of an arrangement of the coils according to an embodiment.

FIG. 4 outlines an example for a possible measurement without a flow according to an embodiment.

FIG. 5 outlines an example for a possible measurement with a flow according to an embodiment.

FIG. 6 depicts an example of a flow-measurement according to an embodiment.

### DETAILED DESCRIPTION

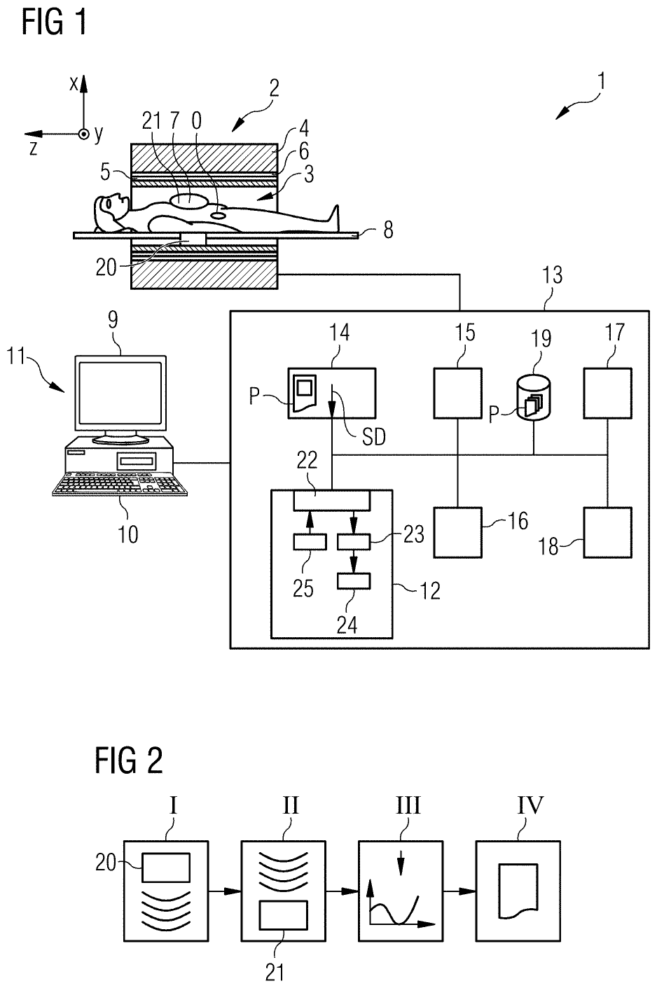

FIG. 1 depicts a schematic representation of a magnetic resonance imaging system 1 (“MRI system”). The MRI system 1 includes the actual magnetic resonance scanner (data acquisition unit) 2 with an examination space 3 or patient tunnel in which a patient or test person is positioned on a driven bed 8, in whose body the actual examination object is located.

The magnetic resonance scanner 2 may be equipped with a basic field magnet system 4, a gradient system 6, as well as an RF transmission antenna system 5, and an RF reception antenna system 7. In the depicted embodiment, the RF transmission antenna system 5 is a whole-body coil permanently installed in the magnetic resonance scanner 2, in contrast to which the RF reception antenna system 7 is formed as a plurality of local coils to be arranged on the patient or test subject. The whole-body coil may also be used as an RF reception antenna system, and the local coils may respectively be switched into different operating modes.

The RF reception antenna system 7 is used as RF antennae 21. Below the object, a generator coil 20 is located. In another example, the RF antennae 21 may be at the position of the whole-body coil (or the whole-body coil may be used if it includes more than one single coil), and the local coil 7 may be used as generator coil 20.

The basic field magnet system 4 is configured such that a region of Interest (“RoI”) may be recorded. Here it is configured in a typical manner so that it generates a basic magnetic field in the longitudinal direction of the patient, i.e. along the longitudinal axis of the magnetic resonance scanner 2 that proceeds in the z-direction. The gradient system 6 may include individually controllable gradient coils in order to be able to switch (activate) gradients in the x-direction, y-direction, or z-direction independently of one another.

The MRI system 1 depicted is a whole-body system with a patient tunnel into which a patient may be completely introduced. However, embodiments may also be used at other MRI systems, for example with a laterally open, C-shaped housing, as well as in smaller magnetic resonance scanners in which only one body part may be positioned.

Furthermore, the MRI system 1 includes a central control device 13 that is used to control the MRI system 1. The central control device 13 includes a sequence control unit 14 for measurement sequence control. With the sequence control unit 14, the series of radio frequency pulses (“RF pulses”) and gradient pulses may be controlled depending on a selected pulse sequence or, respectively, a series of multiple pulse sequences to acquire magnetic resonance images of the RoI within a measurement session. For example, such a series of pulse sequences may be predetermined within a measurement or control protocol P. Different control protocols P for different measurements or measurement sessions may be stored in a memory 19 and may be selected by an operator (and possibly modified as necessary) and used to implement the measurement.

To output the individual RF pulses of a pulse sequence, the central control device 13 includes an RF transmission device 15 that generates and amplifies the RF pulses and feeds the RF pulses into the RF transmission antenna system 5 via a suitable interface (not shown in detail). To control the gradient coils of the gradient system 6, the control device 13 includes a gradient system interface 16. The sequence control unit 14 communicates in a suitable manner with the RF transmission device 15 and the gradient system interface 16 to emit the pulse sequence.

Moreover, the control device 13 includes an RF reception device 17 (likewise communicating with the sequence control unit 14 in a suitable manner) to acquire magnetic resonance signals (i.e. raw data) for the individual measurements, by which magnetic resonance signals are received in a coordinated manner from the RF reception antenna system 7 within the scope of the pulse sequence.

A reconstruction unit 18 receives the acquired raw data and reconstructs magnetic resonance image data therefrom for the measurements. The reconstruction may be performed on the basis of parameters that may be specified in the respective measurement or control protocol. For example, the image data may then be stored in a memory 19.

Operation of the central control device 13 may take place via a terminal 11 with an input unit 10 and a display unit 9, via which the entire MRI system 1 may thus also be operated by an operator. MR images may also be displayed at the display unit 9, and measurements may be planned and started by the input unit (for example, in combination with the display unit 9), and suitable control protocols may be selected (and possibly modified) with suitable series of pulse sequences as explained above.

The control device 13 includes a system 12 configured to perform the method for measuring blood flow. The system 12 includes the following components that may be software modules. The system includes:

A first data-interface 22 configured for sending a signal to a generator coil 20. The signal is configured to generate a (low power) modulated magnetic field in the generator coil 20. The signal may be produced by a generator unit 25. Since the recording RF antennae include an Eigenfrequency in the range of MHz, the signal may be a periodic MHz-signal.

A second data-interface 22 configured for recording response signals of the modulated magnetic field from the object measured with an RF reception system. At least the response signals are measured by two RF antennae of the RF reception system. The first and second data interface are one single data interface configured for bi-directional communication.

A separation unit 23 configured to separate a flow component of the recorded signals from the signal components resulting from cardiac and respiratory motion. Since the measured signals may be turned into digital values by an analog-digital-discriminator, the separation may be performed by calculating steps, e.g. by a calculation based on an Independent Component Analysis.

A computing unit 24 that is configured to compute parameters of the measured flow. A value for the flux (blood volume per second) and the periodicity of the blood flow is calculated based on the separated data. The flux may be determined from the phase shift between the signals of two (or more) antennae, the volume of flowing blood by the amplitude of a signal.

The generator coil 20 is part of the magnetic resonance scanner 2 or may be regarded as a part of the system 12. The same is valid for the RF antennae 21. The local coils 7 of the magnetic resonance scanner 2 are used as RF antennae 21. However, also the RF antennae 21 may be regarded as a part of the system 12.

The MRI system 1 and the control device 13 may include a number of additional components that are not shown in detail here but may be present in such systems, for example a network interface in order to connect the entire system with a network and be able to exchange raw data and/or image data or, respectively, parameter maps, but also additional data (for example patient-relevant data or control protocols).

FIG. 2 depicts a block diagram of the process flow of a method for measuring blood flow.

In step I, a modulated magnetic field is generated by a generator coil. The modulation may be following an alternating signal. The frequency of the modulation of the magnetic field is given with the field-strength of the predefined basic magnetic field. The field-strength defines the Larmor frequency that is measured during medical imaging. The Eigenfrequency of the RF reception antenna system 7 is configured to meet the Larmor frequency. The frequency of the modulation may be higher or lower than the Larmor frequency but lie within the bandwidth of the RF reception antenna system 7. The frequency of the modulation may be between 22 MHz and 125 MHz. However, depending on the magnetic field, the frequency may be higher or lower.

In FIG. 1, the generating coil 20 is positioned near the object O. The modulated magnetic field is generated near the region of interest, since the field-strength of the modulated magnetic field decreases with increasing distance d with approximately 1/d³ (Biot-Savart law).

In step II, response signals of the modulated magnetic field from the object are recorded with an RF reception system, e.g. as shown in FIG. 1 the local coil 7. The response signals may be measured by at least two RF antennae of the RF reception system.

The modulated magnetic field applied in step I induces eddy currents in the blood that is a conductive fluid. The eddy currents in turn generate a magnetic field opposing the generated field. When the blood moves due to the heart's pumping activity an additional current is induced due to motion and the local magnetic environment is changed. This may be recorded by the RF antennae.

In step III, the flow component of the recorded signals is separated from the signal components resulting from movements of the object, such as cardiac and respiratory motion. Due to the pumping action of the heart, the signals from the blood flow may be identified and separated from other contributions to the signal by regarding the shape and/or other (statistical) parameters (as e.g. frequency or shape of the distribution) of the signal. For separation from contributions from cardiac and respiratory motion, appropriate blind and/or semi-blind source separation methods may be used such as e.g. ICA.

In step IV, parameters of the blood flow are computed, such as a value of the flux and/or the periodicity of the flow.

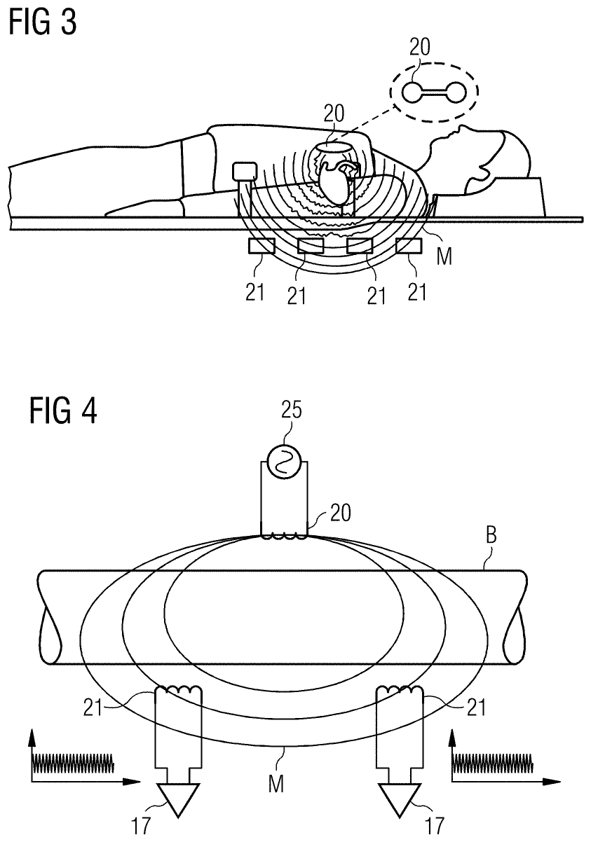

FIG. 3 depicts an example of an arrangement of the coils. Unlike FIG. 1, where a local coil 7 was used as RF antenna 21, the local coil 7 is now used as generator coil 20 sending a modulated magnetic field M with a frequency of about 50 MHz to the heart of a patient. The generator coil 20 may be dumbbell-shaped as depicted in the dashed example. The movement of the heart may be measured as well as the flow in the Aorta.

Several RF antennae 21, that may be part of the RF reception antenna system 7, measure the response of the modulated magnetic field after it has passed heart and aorta. Additional antennae may be used, too, e.g. RF antennae in the body array, since the response signal is emitted by the object in all directions.

The signals recorded with the RF antennae 21 may then be processed in that the movement of the heart is separated from the contribution of the blood flow in the aorta as already explained above.

FIG. 4 outlines an example for a possible measurement without a flow. A blood vessel B lies between a generator coil 21 on one side and two RF antennae 20 on the other side. The RF antenna 20 emits a modulated magnetic field M that is created in a generator unit 25 with a frequency in the order of some ten to some hundred MHz, depending on field-strength. The RF antennae 21 measure the modulated magnetic field M passing through the blood vessel B and an RF reception device 17 at each RF antenna forms a measuring signal or measurement data. The measured signal of each RF antenna 21 is depicted adjacent the respective RF antenna 21. Since there is no flow in the blood vessel B in the figure, the signals are almost identical (for example, due to the symmetric setup of the RF antennae 21). the generator coil may e.g. also lie between the blood vessel and the RF antennae, since it is not necessary that the blood vessel lies between the generator coil and the RF antennae.

FIG. 5 outlines an example for a possible measurement with a flow. The same setup is used as in FIG. 4. In contrast to FIG. 4, there is now blood flowing through the blood vessel B causing the magnetic field to slightly deform in comparison to FIG. 4. Due to the deformation, the measured signals of the RF antennae 21, again depicted adjacent the respective RF antenna 21, are different. The difference offers a possibility to separate the contribution of the blood flow from contribution of other measurements (that may e.g. form an identical offset in both measurements) and a possibility to calculate parameters of the flow itself (e.g. the flux or the periodicity).

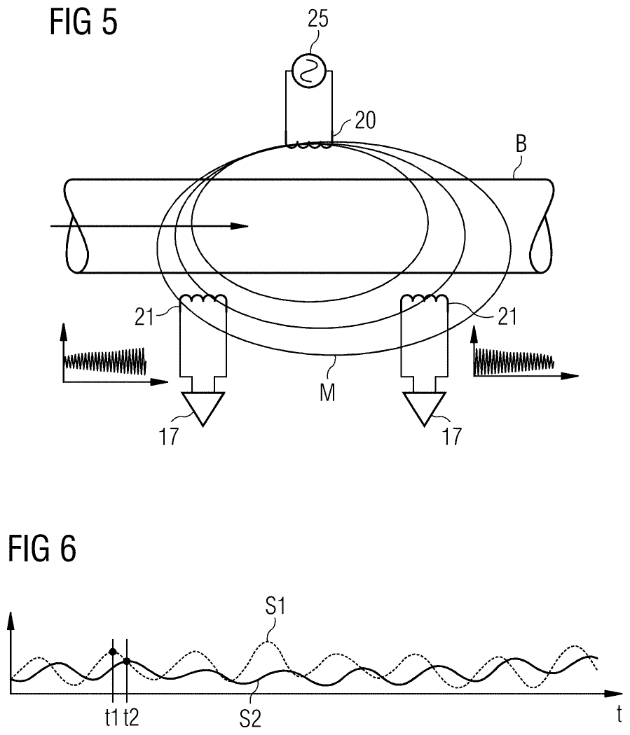

FIG. 6 depicts an example of a flow-measurement, e.g. recorded with the setup of FIG. 5. The amplitude of the measured signals S1, S2 is drawn against the time tin arbitrary units (e.g. mV). The difference between the amplitudes of the two signals S1, S2 results from different energy input in the RF antennae 21 (see e.g. the different field strength in FIG. 5 at the position of the RF antennae 21). Thus, the output of the RF antennae 21 depends on their position relative to the generator coil 20 and the flow direction.

It may be seen that the heartbeat modulates the signals S1, S2 so that the pulse frequency of the blood vessel B may be determined. Furthermore, the signal in the second coil is delayed that may be seen by comparing the two points in time t1, t2 pointing to respective peaks of the blood flow. The delay may e.g. be approximately 120 ms. Since the position of the RF antennae 21 is known, the delay may be used to calculate further parameters as e.g. the velocity of the flow.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for measuring blood flow, the method comprising:
generating a modulated magnetic field by a generator coil;
recording response signals of the modulated magnetic field from an object with an RF reception system, wherein the response signals are measured by at least two RF antennae of the RF reception system; and
separating a flow component of the recorded response signals from signal components resulting from movements of the object.

2. The method of claim 1, wherein the generator coil is embedded in a magnetic resonance scanner of a magnetic resonance imaging system.

3. The method of claim 2, wherein the generator coil is embedded in an RF reception antenna system of the magnetic resonance scanner.

4. The method of claim 2, wherein the RF reception system is part of the magnetic resonance imaging system, wherein the response signals of the modulated magnetic field are recorded by RF antennae from the RF reception antenna system of the magnetic resonance scanner.

5. The method of claim 1, wherein more than three RF antennae are used for measurement of the response signals.

6. The method of claim 1, wherein the separation of the flow component of the recorded signals from signal components resulting from movements of the object comprises using blind or semi-blind source separation methods.

7. The method of claim 6, wherein separation uses Independent Component Analysis (ICA) or a machine learning algorithm trained with ground truth data.

8. The method of claim 1, wherein the object is arranged between the generator coil and the RF antennae; and wherein at least the signals from the two RF antennae of the RF reception system positioned closest to a line parallel to the blood flow intended to be measured is recorded.

9. The method of claim 1, wherein a modulation of the modulated magnetic field includes a frequency within a frequency range that corresponds to a Larmor frequency of hydrogen atoms of the object in an applied magnetic field within a variance of less than 10 percent deviating from the Larmor frequency or within a receiving frequency bandwidth of the RF antennae used for the measurement or within a frequency range of less than 10 percent deviating from an Eigen frequency of the RF antennae.

10. The method of claim 1, further comprising computing parameters of a flow including a value of a flux or a periodicity after separating the flow component of the recorded signals.

11. A system for measuring blood flow, the system comprising:
a first data-interface configured for sending a signal to a generator coil, wherein the signal is configured to generate a modulated magnetic field in the generator coil;
a second data-interface configured for recording response signals of the modulated magnetic field from an object measured with an RF reception system, wherein at least the response signals are measured by two RF antennae of the RF reception system; and
a separation unit configured to separate a flow component of the recorded response signals from signal components resulting from movements of the object.

12. The system of claim 11, further comprising a computing unit configured to a value of a flux, a value of a periodicity, or the value of the flux and the value of the periodicity of a measured flow.

13. The system of claim 11, further comprising a generator coil configured to generate a modulated magnetic field, wherein the generator coil is positioned inside a magnetic resonance scanner and the system further comprises a generator unit for producing the modulation signal for the modulated magnetic field.

14. The system of claim 11, further comprising an RF reception system with at least two RF antennae configured to record response signals the modulated magnetic field from the object, wherein at least the response signals are measured by the RF antennae, wherein the RF antennae is an RF reception antenna system of a magnetic resonance imaging system.

15. A non-transitory computer implemented storage medium that stores machine-readable instructions executable by at least one processor for measuring blood flow, the machine-readable instructions comprising:
generating a modulated magnetic field by a generator coil;
recording response signals of the modulated magnetic field from an object with an RF reception system, wherein the response signals are measured by at least two RF antennae of the RF reception system; and
separating a flow component of the recorded response signals from signal components resulting from movements of the object.

16. The non-transitory computer implemented storage medium of claim 15, wherein the generator coil is embedded in a magnetic resonance scanner of a magnetic resonance imaging system.

17. The non-transitory computer implemented storage medium of claim 16, wherein the generator coil is embedded in an RF reception antenna system of the magnetic resonance scanner.

18. The non-transitory computer implemented storage medium of claim 16, wherein the RF reception system is part of the magnetic resonance imaging system, wherein the response signals of the modulated magnetic field are recorded by RF antennae from the RF reception antenna system of the magnetic resonance scanner.

19. The non-transitory computer implemented storage medium of claim 15, wherein more than three RF antennae are used for measurement of the response signals.

20. The non-transitory computer implemented storage medium of claim 15, wherein the separation of the flow component of the recorded signals from signal components resulting from movements of the object comprises using blind or semi-blind source separation methods.
